(19) United States
(12) Patent Application Publication
(10) Pub. No.: US 2006/0099282 A1
(43) Pub. Date: May 11, 2006

(54) Product of coromandel and method for its use

(76) Inventor: Kazuoki Ishihara, Kailua, HI (US)

Correspondence Address: KAZUYUKI NIEDA;DTS, 5171 CLARETON DRIVE, AGOURA HILLS, CA, 91301, US

(21) Appl. No.: 11/029,806

(22) Filed: Jan. 4, 2005

(30) Foreign Application Priority Data
Nov. 10, 2004 (JP) JP2004-354641

(51) Int. Cl. A61K 36/185; A61K 35/74

(52) U.S. Cl. 424/773; 424/93.45; 424/195.16

(57) ABSTRACT

The present invention relates to a product of coromandel (Asystasia gangetica) and its use as a food by-product and medical value. By extracting the leaves of the coromandel (an antidiabetic agent or lactic acid bacteria promoter) for use in foods and animal feeds and dried goods, the manufacturing of a food and drinks supplement can produce an anti-diabetic effect, growth promoting effect to lactic acid bacteria and food flavor-enhancing effect. Five examples of extraction, production and recommended methods are presented.

### BACKGROUND OF THE INVENTION

The present invention relates to a product of coromandel (Asystasia gangetica) and a method for use.

Coromandel is a plant distributed in subtropical and tropical regions, and in parts of Africa. The leaves are eaten as food or used as folk medicine for stomach pain or asthma. Anti-asthmatic property of hexane, ethylacetate and methanol extracts of the leaves of this plant are reported (Journal of Ethnopharmacology, Vol. 89(1), pp. 25-36, 2003); but to date no industrial use has been made or reported.

### SUMMARY OF THE INVENTION

The present inventors have found that an aqueous extract component of coromandel leaves, abbreviated as “the component” hereinafter, reduces the blood sugar of experimental diabetic animals. The component accelerates the proliferation of lactic acid bacteria, and improves the flavor of foods and produce a novel aroma.

The present invention incorporated the following: (1) the component; (2) a method for manufacturing the component by said method; (3) an augmentation to food, drink, pharmaceutical drug, feed, spice, or pharmaceutical drug for animals, which is used to prevent or treat diabetes or to accelerate proliferation of lactic acid bacteria in the intestines; and (4) to facilitate the production of a yogurt, a manufacturing method in which the component is added to the starting materials, or a starter containing the component or a lactic acid bacteria proliferation promoter to which the component is readily added.

### DETAILED DESCRIPTION OF THE INVENTION

The component is a useful substance that is readily used in foods, drinks, or a pharmaceutical drug that produces an anti-diabetic and lactic acid bacteria proliferation-promoting effects. Examples of production for the component are described as follows. Coromandel leaves are collected, washed with water, placed in an amount of boiling water approximately 5-fold the weight of the coromandel leaves, maintained for 10 minutes, and filtered; the filtrate is freeze-dried, and the component, comprising approximately 3% of the weight of the coromandel leaves, is obtained. When this material is made into a powder, it is pale brown in color and readily soluble in water with very slight or no perceptible odor. When dissolved in water, this material forms a pale brown solution including yellow or green. Addition to food or drink is easy, the flavor of the original food or drink is infrequently impaired, and use is easy. Additionally, by replacing coromandel leaves in lieu of water in cow milk, soy milk, juices, edible vinegars, or other liquids including copious water and then heating or steeping this material for a long duration, the component can be released into such foods creating an improvement in food flavor by reducing the distinctive odor and enhancing the sweetness of soy milk, or lessening the pungent odor of edible vinegars and thereby creating a desirable fragrance.

The antidiabetic effect and lactic acid bacteria proliferation-promoting effect of the present extract is described hereafter.

### EXAMPLE 1

Antidiabetic effect. 4-week-old BALB/c mice were raised prefatorily 1 week, after which 50 mg/kg body weight streptozotocin was injected into the caudal vein, and 72 hours thereafter, mice with a blood glucose value of 11 mmol/l or higher were divided into a control group and an administration group. The administration (treatment) group comprising of 5 mice were given an oral probe daily, for intragastric, of 10 mg of the component (dissolved in 1 ml physiological saline). The control group received 1 ml physiological saline. Blood glucose levels were observed on Day 14 and Day 28. Blood collected at the time of blood glucose observation reflected fasting for the prior 12 hours. Results from a t-test showed a statistically significant difference between administration and control groups on blood glucose in that the Blood glucose measures in the administration group declined (See Table 1). Alpha level was set a priori at 0.05.

TABLE 1
Effect of the component on mouse blood glucose

| | Day 0 | Day 14 | Day 28 |
|---|---|---|---|
| Control group | 15.4 ± 2.66 | 17.1 ± 3.26 | 17.8 ± 3.24 |
| Administration group | 15.6 ± 1.82 | 15.1 ± 0.73 | 13.9 ± 0.70* |

Figures are mean ± standard deviation blood glucose values (mmol/l).
*Significant difference versus controls at 5% or lower critical level

### EXAMPLE 2

Lactic acid bacteria proliferation-promoting effect. The component was added to commericial ultra high temperature sterilized (2 seconds at 130° C.) cow milk at ratios of 0, 65, 130, and 260 mg/l concentrations. A commercial starter for yogurt manufacture (Trade name DPLABY-2C, Kyowahaihuzu Co., Ltd.) was added at a ratio of 10 mg/l, and the material was fermented at 42° C. The time required until coagulation is as shown in Table 2.

TABLE 2
Effect of the component on fermented milk coagulation time

| Added amt. (mg/l) | Time to coagulation |
|---|---|
| 0 | 10 hours 50 minutes |
| 65 | 9 hours |
| 130 | 5 hours 50 minutes |
| 260 | 5 hours 20 minutes |

Fermentation time was shortened markedly at 130 mg/l and higher concentrations of the component. Table 3 presents the results obtained from investigation of time to coagulation using varying added amounts of the same starter.

TABLE 3
Starter concentration and time to coagulation

| Starter | | | |
|---|---|---|---|
| concentration | Component amt (mg/l) | | |
| (mg/l) | 0 | 130 | 260 |
| 1 | 16 hours | 10 hours 10 minutes | 8 hours 10 minutes |
| 10 | 10 hours 50 | 5 hours 50 minutes | 5 hours 20 minutes |
| | minutes | | |
| 50 | 7 hours 30 | | |
| | minutes | | |
| 100 | 5 hours 50 | 4 hours 50 minutes | 4 hours |
| | minutes | | |

These results show that when cow milk coagulation time was determined, addition of the component allowed reduction of the amount of starter used to one-tenth or lower. As described above, use of the component facilitated the production of a yogurt and allowed a reduction of the amount of an expensive starter used. Additionally, yogurt to which the component was added demonstrated no difference in flavor, texture, or color from one without addition

The same was also true in the case of use of a commercial yogurt as a starter. 0.1 ml Aloe Yogurt (Morinaga Milk Industry Co., Ltd.) was added to 1 liter commercial ultra high temperature sterilized milk, and the material was maintained at 42° C. Comparison of the coagulation time of milk without addition to that of milk to which 130 or 260 mg of the component was added to 1 liter showed that addition of the component shorted the coagulation greatly, from 10 hours, 40 minutes in milk without addition, to 7 hours, 10 minutes in milk with 130 mg addition, and 6 hours, 40 minutes in milk with 260 mg addition. In ordinary households, yogurts are often produced from cow milk with a commercial yogurt as a starter, but because this activity was not carried out by specialists or technicians, which is frequently the case that a yogurt cannot be achieved with good results. Domination by admixed bacteria sooner than lactic acid bacteria is one principal cause of failure, and to the extent that there is rapid activity and proliferation of lactic acid bacteria and completion of fermentation in a short duration, failure will be assured as infrequent. The component was also useful in domestic yogurt production as described.

### EXAMPLE 3

The lactic acid bacteria proliferation-promoting effect of the component is exhibited not only among yogurt-producing bacteria; it is also seen in the same fashion among indigenous intestinal lactic acid bacteria. As shown in Table 4, comparison of proliferation of Enterococcus faecium, a representative, indigenous intestinal lactic acid bacteria also used in intestinal medications showed in culture media including and not including the component that the component accelerated proliferation of the bacteria markedly. A culture medium including 0.5% peptone, 0.1% potassium dihydrogenphosphate, 0.1% dipotassium hydrogen phosphate, and 0.3% glucose was adjusted to pH 6.8; 0, 0.1, 0.02, and 0.05% of the component was added; and the material was autoclaved at 121° C. and sterilized for 15 minutes to the create test medium. Enterococcus faecium NBRC 100602 isolated from humans (obtained from the National Institute of Technology and Evaluation) was cultured 24 hours in a basic culture medium not including the component, each test medium was inoculated with 1/10,000 of this material, the material was cultured at 37-40° C., and absorbance (turbidity) at 660 nm was observed over time with a spectrophotometer.

TABLE 4
Proliferation-promoting effect of the component on
intestinal lactic acid bacteria(E. faecium)

| | Culturing time | | | |
|---|---|---|---|---|
| Component amount (%) | 3 hours | 6 hours | 12 hours | 24 hours |
| 0 | 0.04^{a)} | 0.13 | 0.27 | 0.31 |
| 0.01 | 0.10 | 0.18 | 0.29 | 0.34 |
| 0.02 | 0.14 | 0.26 | 0.32 | 0.36 |
| 0.05 | 0.18 | 0.31 | 0.35 | 0.38 |

^{a)}increase of absorbance at 660 nm

Intake of the component had an anticipated association with increased proliferation of intestinal lactic acid bacteria, inhibition and enhanced elimination of pathogenic bacteria and other harmful microbes, and maintenance of health.

### EXAMPLE 4

There was utility in dried coromandel leaves of a type whereby the component was obtained readily by water, hot water or was dissolved readily in the digestive tract postprandially. The present inventor found that in an instance wherein a dried product of leaves was obtained, rapid heating prior to drying of the leaves was effective, and that in such heating, microwave heating or heating by means of heating metallic sheets was particularly effective. Dry coromandel leaves were produced by microwave heating (10 seconds at 500 watts per 1 gram leaves). Similarly, coromandel leaves were also washed with water or pressed for 5-seconds between metal sheets heated to 120-150° C., and blew hot air at 80° C. were also manufactored. The weight of dry coromandel leaves was approximately 10% the weight of the original coromandel leaves. 150 ml hot water was poured into 1 g dry coromandel leaves to create a type of tea which was drunk. The flavor included sweetness and was determined to be good. The color of the liquid was green and appeared attractive. The leaves also opened attractively and were a vivid and vibrate green. If heating was not carried out prior to drying, the color was weak and astringency was strongly perceptible when hot water was poured.

The leaves also become shriveled and did not open attractively, and their color was not as vivid. If heating prior to drying was carried out by application of steam and steaming, the color of the liquid when hot water was poured grew more intense, the longer the duration of heating. However 5-10 minutes of heating time was needed to achieve an intensity of color equivalent to that when microwave heating has been carried out and hot water poured on the dried leaves. Additionally, a brown rather than green hue prevailed, emanating little sweetness, and the leaves did not open attractively with coloration of brown and little vividness and vibrancy.

### EXAMPLE 5

Preferred embodiments of the present invention assume many forms (1 to 11). When the object was to prevent diabetes or to retain normal intestinal microflora, an item easy to ingest routinely as a food or drink was desirable. An intake of 100 mg or more at one instance, by conversion according to the component, was deemed effective. When used in treatment of diabetes, a capsule or tablet agent form was desirable to allow administration of 1 g or more per 1 day, by conversion according to the component.

Methods of Use

(1) A powder of the component is tightly sealed in an aluminum laminate bag or a glass bottle. At the time of use, the material is taken with a small spoon, 100-200 mg is added to miso soup or a stew, and 10-50 mg is mixed into a single portion of pet food for use. The result was good, with no disagreeable perception whatsoever.

(2) A powder of the component (10% coromandel extract) is mixed with instant coffee grinds to produce instant coffee containing a coromandel extract. The product is used in ordinary fashion. The flavor is good.

(3) 0.1% of the extract is added to unpasteurized cow milk, pasteurization is carried out, and cow milk containing a coromandel extract is obtained.

(4) Dried coromandel leaves are mixed with roasted coffee beans and are also ground in a coffee mill to produce a coffee powder containing coromandel. The product is placed in a drip apparatus as in the case of ordinary coffee, hot water is poured, and the product is drunk. The product is good.

(5) Coromandel leaves washed with water and cut into 2 cm-wide portions are added to soy milk heated to 98-100° C. in the amount of 2% by soy milk weight. The temperature is maintained for 10 minutes, and the product is then filtered immediately to obtain soy milk containing a coromandel extract. Sweetness is increased, and the odor of soy milk is reduced, with good result.

(6) One gram of dried coromandel leaves tightly sealed in an aluminum laminate bag is produced. During use, the envelope is opened, the contents are placed in a cup or the like, 100-200 ml hot water is poured in, and after several minutes the contents are drunk. Dried coromandel leaves are placed in a blender or crumbled by hand, hot water is poured on the material ground to a size of approximately 0.5-2 square centimeters, and the material is filtered by a mesh and then drunk. When hot water is poured, the ground material opens attractively to its original state in a cup or the like, giving an attractive impression, and the material can therefore be drunk without filtering by a mesh or the like. Material ground very finely produced in a coffee mill and mixed with hot water can also be drunk without further treatment. In each case the result is good.

(7) Dried coromandel leaves are made into a powder, and 20 grams lactic acid bacteria (faecalis bacteria powder and acidophilus bacteria powder, 10 grams each, obtained from Amano Enzyme Inc. in each case) and 10 grams bifidobacteria (obtained from Amano Enzyme Inc.) is added to 100 grams thereof and blended. 250 milligrams thereof is packed into a gelatin capsule, and 1-2 capsules are swallowed postprandially.

(8) 500 milligrams of the component in powdered form is packed into a gelatin capsule, and 2 capsules are administered 3 times per day.

(9) 50 grams coromandel leaves are added to 500 milliliters white vinegar, and the material is tightly stoppered and left to stand 1 day or longer. The pungent aroma of acetic acid is reduced and becomes a good, sweet and sour aroma. This vinegar can be used as a flavoring (acidulant), and it is also made good to drink by adding and mixing, with 10 milliliters thereof, 15 grams honey, 3 drops lemon juice, and 150 milliliters hot water.

(10) A sterile, powdered form of the component is obtained by sterilizing the filtrate in an autoclave 15 minutes at 121° C. prior to drying, performing subsequent procedures under sterile conditions, freeze-drying and powdering the material, and sealing the material tightly in a sterile vessel. In yogurt production, when a starter is added to cow milk as a starting material, with lactic acid bacteria serving as a proliferation accelerator, 0.03% of the cow milk is added. In other words, 0.3 grams is added to 1 liter cow milk. To facilitate operations, amounts for use with 100 liters, 300 liters, and 500 liters are each produced separately. Amounts for 0.5 liters and 1 liter are also produced for domestic use.

(11) The component powdered is sterilized by standing 20 minutes at 80-90° C. and cooling, and a starter for use in production of 200 liters yogurt is produced by blending 2 grams powdered starter (DPLABY-2C) with 60 grams of the sterilized, powdered extract, and sealing the material tightly in a sterile container.

1. A dried product of a water extract of coromandel leaves.

2. An anti-diabetes agent wherein coromandel leaves or a water extract of the leaves is an active ingredient.

3. A lactic acid bacteria proliferation-promoting agent wherein coromandel leaves or a water extract of the leaves is an active ingredient.

4. A drink, food, or animal feed where in a water extract of coromandel leaves is contained.

5. A food, drink, or animal feed wherein a coromandel component is contained or flavor is enhanced and improved by adding and then heating or steeping coromandel leaves in a food, drink, or animal feed containing copious moisture.

6. A food, drink, or animal feed characterized by the containing coromandel leaves or a water extract of the leaves, with microbes useful to human or animal health.

7. A food, drink, or animal feed of claim 6 wherein the useful microbe is one or more among lactic acid bacteria, bifidobacterium, butyric acid bacteria, Bacillus natto, eubacterium and yeast.

8. A method for manufacturing a dried product of coromandel leaves, characterized in that coromandel leaves are dried after rapid heating.

9. A dried product of coromandel leaves manufactured according to the method of claim 8 and food, drink, or animal feed wherein the dried product of coromandel leaves is used.

10. A method for manufacturing a fermented food, characterized in that a water extract of coromandel leaves is added to a starting material, and fermentation time is shortened or the amount of a starter used is reduced.

11. A manufacturing method of claim 10 wherein said the fermented food is a fermented milk by lactic acid bacteria.

12. A starter for fermented food use, characterized by the containing a water extract of coromandel leaves.

13. A starter of claim 12 wherein said the fermented food is a fermented milk.
